(19) United States
(12) Patent Application Publication
(10) Pub. No.: US 2026/0216391 A1
(43) Pub. Date: Jul. 30, 2026

(54) MOBILE TERMINAL CAPABLE OF CONVENIENTLY DISINFECTING OUTSIDE, AND DISINFECTION METHOD AND APPARATUS

(71) Applicant: ZHEJIANG GEELY HOLDING GROUP CO., LTD., Hangzhou City, Zhejiang Province (CN)

(72) Inventor: Shufu LI, Hangzhou City, Zhejiang Province (CN)

(21) Appl. No.: 19/144,929

(22) PCT Filed: Oct. 8, 2023
(86) PCT No.: PCT/CN2023/123327

§ 371 Date: Jun. 30, 2025

(30) Foreign Application Priority Data
Dec. 30, 2022 (CN) 202211731508.X

(51) Int. Cl. A61L 2/24; A61L 2/104; G01S 17/06

(52) U.S. Cl. CPC A61L 2/24; A61L 2/104; G01S 17/06; A61L 2202/14

(57) ABSTRACT

A mobile terminal capable of conveniently disinfecting an outside is provided with: a disinfection lamp, configured to output a disinfection light wave of a specific wavelength to the outside to disinfect and sterilize a to-be-disinfected object; a time-of-flight (ToF) sensor, configured to receive at least one of the disinfection light wave or a distance measurement light wave reflected by a to-be-measured object to measure a distance between the to-be-measured object and the mobile terminal; and controller, connected to and controlling the ToF sensor and the disinfection lamp through a circuit.

### CROSS-REFERENCE OF RELATED APPLICATIONS

This application is a National Phase of International Application No. PCT/CN2023/123327, filed on Oct. 8, 2023, which claims priority to Chinese Patent Application No. 202211731508.X entitled “MOBILE TERMINAL CAPABLE OF CONVENIENTLY DISINFECTING OUTSIDE, AND DISINFECTION METHOD AND APPARATUS” filed on Dec. 30, 2022, the entire contents each of which is incorporated herein by reference for all purposes.

### TECHNICAL FIELD

The present disclosure relates to the fields of disinfection technology and mobile communication device technology, in particular to a mobile terminal capable of conveniently disinfecting an outside, a disinfection method and apparatus of a mobile terminal, and a storage medium.

### BACKGROUND

Infectious diseases are caused by the spread of germs or virus. Most of these germs or virus are able to survive on a surface of an object for a longer period of time, this further increases the probability of spreading the disease and affects the live safety of people. Therefore, frequent use of public transportation or the need to communicate with the outside world and attend different occasions, people often contact with tables, chairs, computers or tools in different places, the probability of infection of the germs or virus has increased greatly, this poses a serious threat to the health of people.

Ultraviolet disinfection is a common disinfection method, but is affected by the intensity and time of irradiation. On the one hand, the intensity of irradiation is affected by the distance between the location where the ultraviolet light is generated and the to-be-disinfected object, so the use of ultraviolet light for disinfecting the outside requires consideration of the effect of distance. On the other hand, there are differences in the types of germs or virus living on the surface of different objects, the irradiation intensity and effective time to be used are also different, and the most effective way to disinfection is also different. There is a strong correlation between the ultraviolet disinfection time and efficiency, and the type of object.

A time-of-flight (ToF) depth camera is currently experiencing rapid development in the mobile communication industry, such as iPhone 12 and 13. The principle of three-dimensional (3D) TOF technology is that a ToF sensor use a tiny light wave emitter to emit light or laser, and the generated light will bounce off any object and return to the receiver of the sensor. Based on the time difference between the emission of light and the reflection of light by an object back to the receiver of the sensor, the sensor can measure the distance between the object and the sensor, quickly complete functions such as distance measurement, 3D photography, 3D object identification, and 3D modeling.

Therefore, how to comprehensively utilize the mobile communication device, the medical sterilization equipment, the depth camera, the data software processing and other industry advanced technology, research and development of a use of safe, compliant with regulations, with the ultraviolet disinfection function of the mobile phone, has a high economic and social benefits, but also a need to overcome the technical challenges.

### SUMMARY

In view of this, an object of the present disclosure is to disclose a mobile terminal capable of conveniently disinfecting an outside, a disinfection method and apparatus of a mobile terminal, and a storage medium, and to solve, by setting up a ultraviolet disinfection lamp and a safe method of disinfection on the mobile terminal, the problem of inconvenient carrying of the disinfection device of the existing technology that cannot conveniently carry out sterilization and disinfection of objects in contact with the surroundings, thereby leading to an increase in the probability of transmission of viruses or germs and an increase in the risk of the spread of a disease. Furthermore, by combining the use of the disinfection lamp with the 3D depth image obtained by the ToF sensor and other soft and hard measures, the safety of use is substantially improved, making it possible to realize the convenient ultraviolet disinfection function on a mobile phone.

To solve the technical problems, the present disclosure discloses a mobile terminal capable of conveniently disinfecting an outside, being provided with:
a disinfection lamp, configured to output a disinfection light wave of a specific wavelength to the outside to disinfect and sterilize a to-be-disinfected object;
a time-of-flight (ToF) sensor, configured to receive at least one of the disinfection light wave or a distance measurement light wave reflected by a to-be-measured object to measure a distance between the to-be-measured object and the mobile terminal; and
a controller, connected to and controlling the ToF sensor and the disinfection lamp through a circuit.

In some embodiments, in the mobile terminal, the ToF sensor is a LiDAR-type laser radar including a laser emitter, a receiver, and a ToF processor.

In some embodiments, in the mobile terminal, the disinfection lamp and the ToF sensor are provided independently, the ToF processor is connected to the controller through a first circuit, and the disinfection lamp is connected to the controller through a second circuit.

In some embodiments, in the mobile terminal, the disinfection lamp is integrated in the ToF sensor, and the ToF sensor is connected to and controls the disinfection lamp, the laser emitter and the receiver through the ToF processor, respectively.

In some embodiments, in the mobile terminal, the disinfection lamp and the laser emitter are combined as a single unit.

In some embodiments, in the mobile terminal, a laser generator in the laser emitter is capable of emitting, by pulse modulation, a laser for disinfection with high energy and a laser for distance measurement with low energy, and the laser for disinfection is the disinfection light wave.

In some embodiments, the mobile terminal further includes a power supply component, a display screen, a front camera, a rear camera and a flash lamp, that are connected to the controller and controlled by the controller.

In some embodiments, in the mobile terminal, the flash lamp is integrated in the disinfection lamp, and the controller is connected to and controls the flash lamp and the disinfection lamp through a flash-disinfection processor, respectively.

In some embodiments, in the mobile terminal, the controller is connected to a flash-disinfection integrated lamp through a flash-disinfection processor to control the flash-disinfection integrated lamp to emit the disinfection light wave or flash for photography.

In some embodiments, in the mobile terminal, the disinfection lamp is configured to output ultraviolet light or ultraviolet laser to the outside.

In some embodiments, in the mobile terminal, the disinfection lamp is a pulsed ultraviolet laser generating component or a pulsed ultraviolet light generating component.

In some embodiments, in the mobile terminal, the disinfection lamp is disposed in a preset area next to the front camera or the rear camera.

In some embodiments, in the mobile terminal, the disinfection lamp in the preset area is provided in a form of a plate, a strip or a ring, or a point.

In some embodiments, the mobile terminal is a mobile phone, a smart wearable device, or a satellite communication terminal.

The present disclosure discloses a disinfection method, performed by a mobile terminal and including:
receiving a disinfection instruction; and
controlling, based on the disinfection instruction, a disinfection lamp to output a disinfection light wave of a specific wavelength to an outside to perform disinfection and sterilization, where the disinfection lamp is provided on the mobile terminal.

In some embodiments, the disinfection method further includes:
receiving an image identification instruction; and
invoking, based on the image identification instruction, a time-of-flight (ToF) sensor and at least one camera to obtain a three-dimensional (3D) depth image of a to-be-disinfected object, identifying the 3D depth image to determine a type of the to-be-disinfected object and an optimal distance or range for disinfection, and determining, based on the type of the to-be-disinfected object, a disinfection solution.

In some embodiments, the disinfection method further includes:
receiving a distance measurement instruction; and
controlling, based on the distance measurement instruction, the ToF sensor to output a distance measurement light wave to the outside to measure a real-time distance between the mobile terminal and the to-be-disinfected object.

In some embodiments, the disinfection solution includes at least one of: a disinfection light wave parameter, a disinfection start time, or a duration.

In some embodiments, based on the image identification instruction, the method further includes safe use confirmation including:
confirming, by invoking a front camera, that a disinfection user is an authenticated and authorized user, and determining whether a current state is a safe use state; and
in response to determining that the current state is the safe use state, starting a depth image identification process.

In some embodiments, determining whether the current state is the safe use state includes:
displaying a safe use area for holding the mobile terminal on a display screen of the mobile terminal; and
determining, by the safe use area displayed, whether a palm or fingers of a current user operating the mobile terminal is in the safe use state.

In some embodiments, the safe use state represents that a position of the palm or the fingers of the current user is sensed by a position sensing sensor provided on a surface of the mobile terminal to confirm that the palm or the fingers is in a safe use position, and the position sensing sensor is connected to the controller.

In some embodiments, controlling the disinfection lamp to output the disinfection light wave of the specific wavelength to the outside to perform disinfection and sterilization includes:
determining whether the to-be-disinfected object is in the optimal distance or range of the disinfection lamp; and
in response to the to-be-disinfected object being in an effective area of the disinfection lamp, controlling the disinfection lamp to output the disinfection light wave of the specific wavelength to the outside to perform disinfection and sterilization.

In some embodiments, determining whether the to-be-disinfected object is in the effective area of the disinfection lamp includes:
displaying, by invoking the camera, the effective area of the disinfection lamp and a real-time video image of the to-be-disinfected object on the display screen of the mobile terminal, where an icon of the effective area is displayed in the real-time video image of the to-be-disinfected object; and
determining, by the effective area of the disinfection lamp displayed, whether the to-be-disinfected object is in the effective area of the disinfection lamp.

In some embodiments, determining whether the to-be-disinfected object is in the effective area of the disinfection lamp includes:
determining, by a distance sensor, a mid-air distance between the to-be-disinfected object and the mobile terminal; and
determining, by the mid-air distance, whether the to-be-disinfected object is in the effective area of the disinfection lamp.

In some embodiments, determining whether the to-be-disinfected object is in the effective area of the disinfection lamp further includes: invoking the camera to obtain a real-time image, and comparing the real-time image with a 3D depth image of the to-be-disinfected object, identifying whether there is an abnormal object, and in response to there being the abnormal object, the disinfection lamp stops outputting the disinfection light wave, where the abnormal object is a human limb capable of being exposed to a disinfection area, and the human limb includes: a palm, fingers, toes, and a face.

In some embodiments, the disinfection method further includes:
in response to the to-be-disinfected object being not in the effective area of the disinfection lamp, outputting an adjustment prompt information.

In some embodiments, the disinfection method further includes:
indicating a progress of disinfection.

The present disclosure provides a non-transitory storage medium, storing computer-executable instructions thereon, where the computer-executable instructions, when executed by a processor, cause the processor to perform the disinfection method of any one of the embodiments.

### BRIEF DESCRIPTION OF THE DRAWINGS

To describe the technical solutions of the embodiments of the present disclosure more clearly, the following briefly introduces the accompanying drawings required for describing the embodiments. Apparently, the accompanying drawings in the following description show only some embodiments of the present disclosure, and a person of ordinary skill in the art may still derive other drawings from these accompanying drawings without inventive efforts.

FIG. 1 is a structural schematic diagram illustrating a disinfection lamp and a ToF sensor provided independently according to an embodiment of the present disclosure.

FIG. 2 is a structural schematic diagram illustrating a disinfection lamp integrated in a ToF sensor according to an embodiment of the present disclosure.

FIG. 3 is a structural schematic diagram illustrating a disinfection lamp and a laser emitter combined as a single unit according to an embodiment of the present disclosure.

FIG. 4 is a structural schematic diagram illustrating a front camera and a display screen according to an embodiment of the present disclosure.

FIG. 5 is a structural schematic diagram illustrating a flash lamp and a disinfection lamp integrated as a single unit according to an embodiment of the present disclosure.

FIG. 6 is a structural schematic diagram illustrating an ultraviolet disinfection integrated lamp according to an embodiment of the present disclosure.

FIG. 7 is a schematic diagram illustrating a disinfection lamp in a form of a ring according to an embodiment of the present disclosure.

FIG. 8 is a schematic diagram illustrating a disinfection lamp in a form of a strip according to an embodiment of the present disclosure.

FIG. 9 is a schematic diagram illustrating a disinfection lamp in another form according to an embodiment of the present disclosure.

FIG. 10 is a schematic diagram illustrating a state in which a terminal is held with a left hand according to an embodiment of the present disclosure.

FIG. 11 is a schematic diagram illustrating a state in which a terminal with a position sensing sensor is held with a left hand according to an embodiment of the present disclosure.

FIG. 12 is a schematic diagram illustrating a state in which a terminal is held with a left hand according to another embodiment of the present disclosure.

FIG. 13 is a schematic diagram illustrating a state in which a terminal is held with a right hand according to an embodiment of the present disclosure.

FIG. 14 is a schematic diagram illustrating a state in which a terminal is held with a right hand according to an embodiment of the present disclosure.

FIG. 15 is a flowchart illustrating a disinfection method of a mobile terminal according to an embodiment of the present disclosure.

FIG. 16 is a flowchart illustrating a disinfection method of a mobile terminal according to another embodiment of the present disclosure.

FIG. 17 is a block diagram illustrating a disinfection apparatus of a mobile terminal according to another embodiment of the present disclosure.

### DETAILED DESCRIPTION OF THE EMBODIMENTS

In order to at least describe the purposes, solutions, and advantages of the embodiments of the present application more clearly, the solutions in the embodiments of the present application are described below in combination with the drawings for the embodiments. It is apparent that the embodiments in the following description are some embodiments but may not be all of the embodiments of the present application. All other embodiments obtained by a person of ordinary skill in the art based on the embodiments of the present disclosure without creative efforts shall fall within the protection scope of the present disclosure.

### Embodiment 1

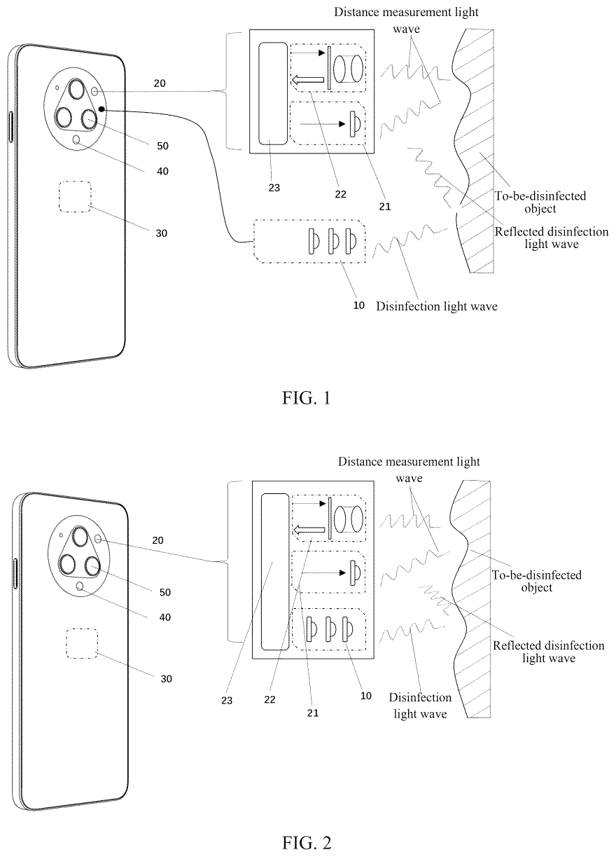

FIG. 1 is a structural schematic diagram illustrating a disinfection lamp and a ToF sensor provided independently according to an embodiment of the present disclosure, and referring to FIG. 1, the mobile terminal of this embodiment is provided with: a disinfection lamp 10, a ToF sensor 20 and a controller 30.

The disinfection lamp 10 is configured to output a disinfection light wave of a specific wavelength to the outside to disinfect and sterilize a to-be-disinfected object. The disinfection lamp 10 in this embodiment is an output component that externally outputs disinfection light waves of a specific wavelength. This embodiment does not limit the specific construction mode of the disinfection lamp 10, as long as it is capable of externally outputting disinfection light waves of a specific wavelength, such as ultraviolet rays, ultraviolet lasers, and the like. For example, an optical lens may be added to the LED light emitting diode lamp as the disinfection lamp 10, or the disinfection lamp 10 may be constructed in a manner that modulates the tri-color phosphor within the LED lamp bead to change the wavelength of the light.

This embodiment does not limit the specific location of the disinfection lamp 10, as long as it can output disinfection light waves of a specific wavelength to the outside. For example, the disinfection lamp 10 may be provided at the back of the mobile terminal, at the front of the mobile terminal, or at the side of the mobile terminal.

This embodiment does not limit the number of disinfection lamps 10, for example, the number of disinfection lamps 10 can be 1, 3, or 4, as long as the disinfection lamp 10 can output disinfection light waves of a specific wavelength to the outside. It is foreseeable that the higher the number of disinfection lamps 10, the higher the disinfection efficiency.

The ToF sensor 20 is configured to receive a disinfection light wave and/or a distance measurement light wave reflected by a to-be-measured object to measure a distance between the to-be-measured object and the mobile terminal. This embodiment does not limit the specific location of the ToF sensor 20, as long as it can detect the distance between the to-be-measured object and the mobile terminal based on the disinfection light wave. For example, the ToF sensor may be provided at the back of the mobile terminal, at the front of the mobile terminal, or at the side of the mobile terminal.

The controller 30 is connected to and controlling the ToF sensor 20 and the disinfection lamp 10 through a circuit. The controller 30 is disposed inside the mobile terminal, and its form and structure are not limited.

In the mobile terminal of this embodiment, the disinfection lamp 10 and the ToF sensor 20 are provided independently, the ToF processor 23 is connected to the controller 30 through a first circuit, and the disinfection lamp 10 is connected to the controller 30 through a second circuit. From an engineering design perspective, adopting this method only requires adding an opening on the surface of the existing rear camera installation area for installing a disinfection lamp, which can be connected to the controller 30 through a second circuit internally. The lowest renovation cost is achieved, but it requires adjusting the existing mobile phone structure layout space.

Furthermore, in the mobile terminal of this embodiment, the ToF sensor 20 is a LiDAR-type laser radar, which includes a laser emitter 21, a receiver 22, and a ToF processor 23. In this embodiment, the laser emitter 21 is used to emit laser light. This embodiment does not limit the type of laser emitted by the laser emitter 21, as long as it can measure the distance of the to-be-measured object and obtain 3D depth images. For example, the laser emitter 21 can emit a laser solely for distance measurement, or a laser that can be used for both distance measurement and disinfection. In this embodiment, the receiver 22 is configured to receive the laser emitted by the emitter, and the ToF processor 23 is configured to calculate the distance between the terminal and the to-be-measured object based on the time when the laser emitter 21 emits the laser and the time when the receiver 22 receives the laser.

In order to improve the accuracy of measurement, under some working conditions, the receiver 22 can collect the disinfection light waves reflected by the to-be-disinfected object, and use the light wave signal data reflected by the disinfection light waves as one of the basic depth image data of the depth camera.

In this embodiment, the distance measurement of the to-be-measured object can be performed by receiving disinfection light waves, and it can also be used to verify the accuracy of the distance measurement of the to-be-measured object by the laser emitter 21. The distance measurement of the to-be-measured object can also be performed by receiving the distance measurement light waves. It is to be noted that in this embodiment, if distance measurement and disinfection are performed on the to-be-disinfected object, the to-be-disinfected object and the to-be-measured object are the same object. If only the to-be-disinfected object is disinfected and sterilized, the to-be-disinfected object and the to-be-measured object may be not the same object.

It is to be noted that in this embodiment, the to-be-disinfected object and the to-be-measured object may be the same object, that is, the ToF sensor 20 first measures the object, and the disinfection lamp 10 then disinfects the object. The to-be-disinfected object and the to-be-measured object may be not the same object.

The mobile terminal provided by the embodiment of the present disclosure can easily disinfect the outside by adding the disinfection lamp 10, the ToF sensor 20 and the controller 30 to the mobile terminal, and by controlling the disinfection lamp 10 to output disinfection light waves, the mobile terminal can easily disinfect the to-be-disinfected object, and at the same time, the mobile terminal can measure the distance to the to-be-measured object, and by outputting ultraviolet rays or ultraviolet lasers, the mobile terminal can disinfect a variety of viruses and bacteria, and by using a pulsed ultraviolet laser generating component or a pulsed ultraviolet ray generating component, it can be convenient for the disinfection lamp 10 to emit the required light waves, reducing energy consumption. By independently providing the disinfection lamp 10 and the ToF sensor 20 in the mobile terminal, the disassembly of the disinfection lamp 10 has been improved, and the convenience of maintenance has been enhanced. By providing a LiDAR-type laser radar, the ToF sensor 20 can accurately measure the distance to the to-be-measured object. By providing the disinfection lamp 10 in the preset area next to the rear camera 50, the disinfection lamp 10 can not affect the normal use of the terminal in the process of disinfection and sterilization, and the settable area of the disinfection lamp 10 can be increased. The risk posed by the spread of viruses and bacteria is reduced, and the user experience of mobile devices that can easily disinfect the outside is improved.

### Embodiment 2

In conjunction with the Embodiment 1 and FIG. 2, FIG. 2 is a structural schematic diagram illustrating a disinfection lamp integrated in a ToF sensor according to an embodiment of the present disclosure.

The disinfection lamp 10 is integrated in the ToF sensor 20, and the ToF sensor 20 is connected to and controls the disinfection lamp 10, the laser emitter 21 and the receiver 22 through the ToF processor 23, respectively.

In this embodiment, the disinfection lamp 10 is integrated in the ToF sensor 20. By integrating the disinfection lamp in the ToF sensor 20, the installation holes of the rear camera can be reduced, the difficulty of engineering installation can be reduced, the integration level can be improved, the modularity level can be enhanced, the difficulty of maintenance and assembly can be reduced, and the degree of universality can be higher. From a control perspective, handing over the control of the disinfection lamp to the ToF processor 23 enables the two components with distance measurement requirements to be processed uniformly, which can improve the processing efficiency of the processor.

This embodiment does not limit the specific position of the disinfection lamp 10 integrated in the ToF sensor 20, as long as the disinfection lamp 10 is disposed in the same plane as the laser emitter 21. For example, the disinfection lamp 10 can be disposed on the left side of the laser emitter 21, on the right side of the laser emitter 21, or above or below the laser emitter 21.

### Embodiment 3

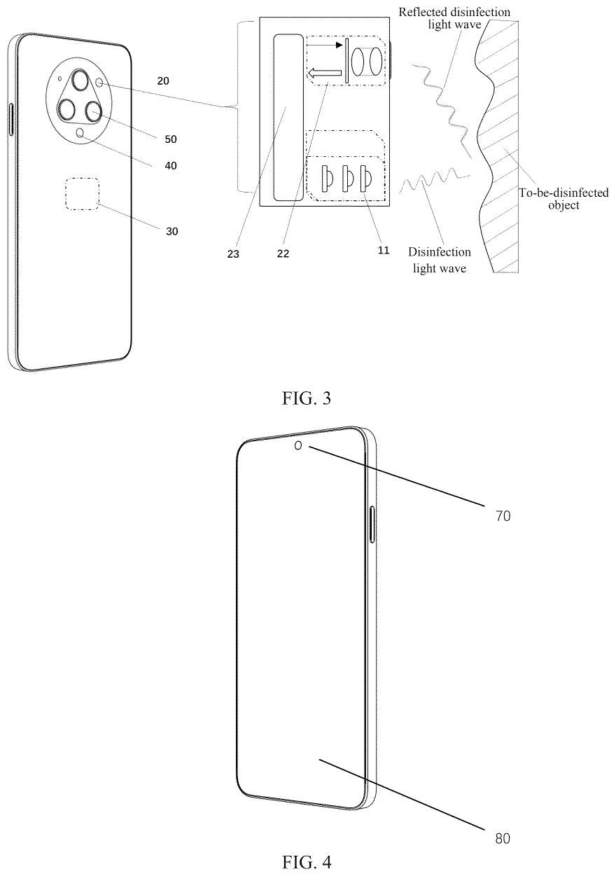

In conjunction with the Embodiment 1 and FIG. 3, FIG. 3 is a structural schematic diagram illustrating a disinfection lamp and a laser emitter combined as a single unit according to an embodiment of the present disclosure.

In the mobile terminal of this embodiment, the disinfection lamp and the laser emitter are combined as a single unit. A laser generator in the laser emitter 21 is capable of emitting, by pulse modulation, a laser for disinfection with high energy and a laser for distance measurement with low energy, and the laser for disinfection is the disinfection light wave.

The disinfection lamp 10 requires ultraviolet rays or ultraviolet lasers or pulsed ultraviolet lasers with high external energy for disinfection, while the ToF sensor 20 is provided with a laser emitter 21. In actual use, through current modulation, the range of laser light and the range of energy density that can be emitted by the laser emitter 21 are increased, so that the laser emitter 21 emits a laser light for disinfection when the disinfection is required, and a low-energy laser light for distance measurement when the distance measurement is required, so that two similar functional components are combined into one, reducing the number of components and improving the degree of integration.

### Embodiment 4

Combining the Embodiment 1 and FIG. 4, in this embodiment, the mobile terminal further includes a power supply component disposed within the mobile terminal (not shown in the figures), a display screen 80, a front camera 70, a rear camera 50, and a flash lamp 40. In other embodiments, the power supply component can also be configured to be disposed in the mobile terminal (not shown in the figures). The display screen 80, the front camera 70, the rear camera 50, and the flash lamp 40 can be disposed in the positions as shown in FIGS. 1 to 3 or other attached figures.

In the related art, the mobile terminal such as a mobile phone generally have a display screen 80, a front camera 70, a rear camera 50, and a flash lamp 40, which belong to the conventional technology and installation mode in this field and are not the focus of the present disclosure. This embodiment will not be repeated.

### Embodiment 5

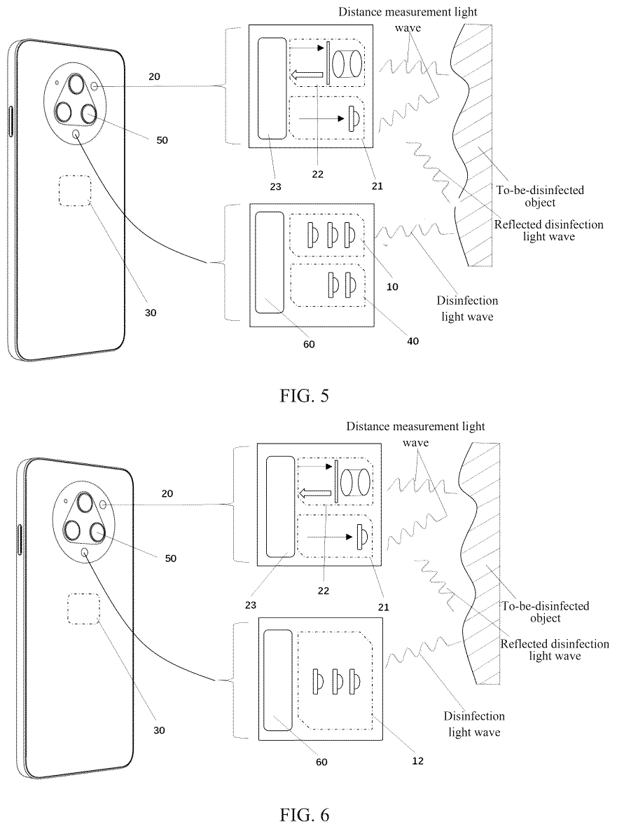

In conjunction with the Embodiment 4 and FIG. 5, FIG. 5 is a structural schematic diagram illustrating a disinfection lamp and a flash lamp combined as a single unit according to an embodiment of the present disclosure.

In the mobile terminal of this embodiment, the flash lamp 40 is integrated in the disinfection lamp 10, and the controller 30 is connected to and controls the flash lamp 40 and the disinfection lamp 10 through a flash-disinfection processor 60, respectively. The flash lamp and the light-emitting part of the disinfection lamp are integrated into a single light-emitting module, or the light emitting diodes (LEDs) that can emit ordinary white light or flash are retained among the plurality of LEDs in the module having the lamps, and they are controlled uniformly by the same control module, i.e., the flash-disinfection processor 60.

In this embodiment, the flash lamp 40 is integrated into the disinfection lamp 10, and the installation position using the original position for the flash lamp 40 may require a larger opening to reduce the number of installation openings for the rear camera, lower the difficulty of engineering installation, improve integration, enhance modularity, reduce the difficulty of maintenance and assembly, and achieve higher universality. From a control perspective, the control of the disinfection lamp is handed over to the flash-disinfection processor 60 so that the two components with needs of light emitting to the outside are controlled in unison, which can improve the processing and control efficiency of the processor.

It is to be noted that this embodiment does not limit the specific position of the flash lamp 40 next to the disinfection lamp 10. For example, the flash lamp 40 can be disposed on the left side of the disinfection lamp 10, on the right side of the disinfection lamp 10, or above or below the disinfection lamp 10.

### Embodiment 6

In conjunction with the Embodiment 5 and FIG. 6, furthermore, in order to improve the integration of components of the mobile terminal that can conveniently disinfect the outside and reduce the cost of disinfection.

In the mobile terminal of this embodiment, the controller 30 is connected to a flash-disinfection integrated lamp 12 through a flash-disinfection processor 60 to control the flash-disinfection integrated lamp 12 to emit the disinfection light wave or the flash for photography.

It is to be noted that this embodiment does not limit the number of integrated the flash lamp 40 and the disinfection lamp 10. For example, one flash lamp 40 and one disinfection lamp 10 can be integrated, or one flash lamp 40 and three disinfection lamps 10 can be integrated.

This embodiment is a further optimized development of embodiment 5, which enables the same lamp to emit white light for supplementary lighting, and control the lamp to emit ultraviolet light of different wavelengths through current modulation. Ultraviolet-C (UVC) LED, also known as deep ultraviolet C-band LED, can be selected for use, which is the shortest wavelength and highest energy band, and has practical effects on sterilization and disinfection. In practical use, existing technologies have various modulation schemes for ultraviolet light bands, which will not be repeated in this embodiment.

It can be seen that the mobile terminal provided in this embodiment, which can conveniently disinfect the outside, integrates the disinfection lamp 10 into the ToF sensor 20, reducing the space occupied by the disinfection lamp 10 on the mobile terminal and facilitating centralized management. The disinfection lamp 10 is provided on the left, right or both sides of the front camera on the mobile terminal, such that the to-be-disinfected object is conveniently disinfected. The disinfection lamp 10 is provided on the left, right or both sides of the front camera, such that the blocking of the disinfection lamp 10 is it effectively avoided, and the occupied space is reduced. The flash lamp 40 and the disinfection lamp 10 are integrated, such that the cost of the mobile terminal capable of conveniently disinfecting the outside is reduced, and the disinfection efficiency of the disinfection lamp 10 is ensured. The controller 30 is connected with the disinfection lamp 10 through the drive circuit of the flash lamp 40, which improves the integration of the circuit connection, improves the disinfection and sterilization effect of viruses or bacteria, and the risk of the infection caused by viruses and bacteria is reduced, and the experience of the user using the mobile terminal capable of conveniently disinfecting the outside is improved.

### Embodiment 7

In conjunction with the Embodiment 1, in the mobile terminal of this embodiment, the disinfection lamp 10 is configured to output ultraviolet rays or ultraviolet lasers to the outside. The disinfection lamp 10 is a pulsed ultraviolet laser generating component or a pulsed ultraviolet ray generating component. Due to the inherent energy limitation of the mobile terminal, the use of pulsed or intermittent lasers can significantly reduce energy loss.

For the ultraviolet lasers, a laser emitter prepared based on the principle of 355 nm ultraviolet nanosecond laser or femtosecond laser can be used. Existing femtosecond lasers do not use monochromatic light, but use a combination of continuously changing wavelengths distributed around the center wavelength, utilizing their space to achieve femtosecond level pulse output. Femtosecond laser has a fire extinguishing effect on various viruses, including tobacco mosaic virus, human immunodeficiency virus and other pathogens. The principle of killing bacteria is the same as ultraviolet disinfection. It can break the hydrogen bonds and hydrophobic bonds in the protein shell of virus particles, causing partial protein separation or aggregation of virus capsid and envelope proteins, ultimately leading to virus inactivation.

### Embodiment 8

In conjunction with Embodiment 5, the mobile terminal is a mobile phone, a smart wearable device, or a satellite communication terminal.

In the mobile terminal of this embodiment, the disinfection lamp 10 is disposed in a preset area next to the front camera or the rear camera 50.

This embodiment does not limit the specific position of the disinfection lamp 10. For example, the disinfection lamp 10 may be above the front camera or rear camera 50, below the front camera or rear camera 50, on the left side of the front camera or rear camera 50, on the right side of the front camera or rear camera 50, or any combination of the above positions. This embodiment does not limit the distance between the disinfection lamp 10 and the front or rear camera 50 of the mobile terminal capable of conveniently disinfecting an outside, for example, the distance can be 10 millimeters or 20 millimeters.

Furthermore, in order not to affect the normal use of the terminal by the user during the implementation of disinfection and sterilization, the area of the settable area of the disinfection lamp 10 can be increased. The camera can be the rear camera 50, and the disinfection lamp 10 can be disposed in the preset area beside the rear camera 50. In this embodiment, the preset area next to the rear camera 50 in the mobile terminal is provided with the disinfection lamp 10. It is to be noted that this embodiment does not limit the specific location area of the preset area, for example, it can be one half of the back area near the rear camera 50 of the mobile terminal capable of conveniently disinfecting the outside, or one quarter of the back area near the rear camera 50 of the mobile terminal capable of conveniently disinfecting the outside, or it can be all the back area near the rear camera 50 of the mobile terminal capable of conveniently disinfecting the outside. In this case, a fixed bracket is needed to be used to support the terminal to implement disinfection and sterilization.

Furthermore, the disinfection lamp 10 in the preset area is provided in a form of a plate, a strip or a ring, or a point.

This embodiment does not limit the shape of the disinfection lamp 10, for example, it can be a long strip, a circle, or a square. This embodiment does not limit the distance between the disinfection lamp 10 and the camera of the mobile terminal, for example, the distance can be 2 millimeters or 5 millimeters. This embodiment does not limit the number of disinfection lamps 10 disposed on the left and right sides of the front camera, for example, it can be 1 or 2.

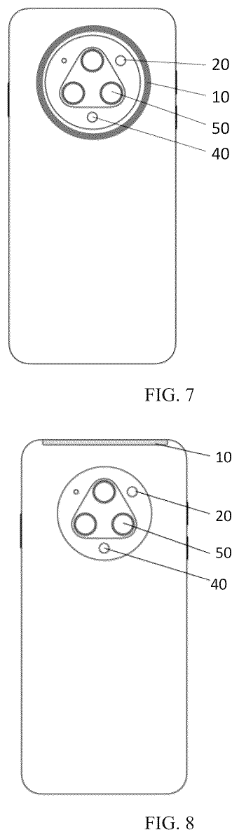

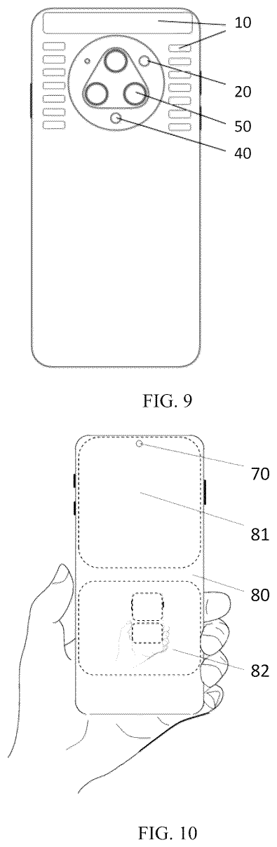

Furthermore, in order to improve the flexibility of providing the disinfection lamp 10, the disinfection lamp 10 in the preset area is provided in a form of a plate, a strip or a ring, or a point. Of course, the disinfection lamp 10 may also be any combination of these forms, as shown in FIGS. 7 to 9 for details. FIG. 7 is a schematic diagram illustrating a disinfection lamp in a form of a ring. FIG. 8 is a schematic diagram illustrating a disinfection lamp in a form of a strip. FIG. 9 is a schematic diagram illustrating a disinfection lamp in another form.

It is to be noted that the disinfection lamp 10 can be in the shape of the plate, a strip or a ring, a point, or any combination of the above shapes.

This embodiment does not limit the effective area of the disinfection lamp 10 in the shape of a plate, for example, it can be half of the back area of the mobile terminal capable of conveniently disinfecting an outside, or one fourth of the back area of the mobile terminal capable of conveniently disinfecting an outside.

This embodiment does not limit the specific number of disinfection lamps 10 in the shape of the strip, for example, it can be 1, 2, or 3.

This embodiment does not limit the specific number of rings of the disinfection lamp 10 in the shape of the ring, for example, it can be 1 ring, 2 rings, or 3 rings.

This embodiment does not limit the arrangement of the disinfection lamp 10 in the shape of the strip, for example, it can be arranged horizontally, vertically, or diagonally. This embodiment does not limit the arrangement of the circular disinfection lamp 10, for example, a large ring over a small ring, or a side-by-side arrangement of rings. This embodiment does not limit the arrangement of the combination of disinfection lamp 10 in the shape of the strip and the disinfection lamp 10 in the shape of the ring, for example, the disinfection lamp 10 in the shape of the strip is disposed inside the disinfection lamp 10 in the shape of the ring, or that the disinfection lamp 10 in the shape of the strip is disposed next to the disinfection lamp 10 in the shape of the ring.

This embodiment does not limit the number of disinfection lamps 10 in the shape of the point. For example, it can be 1 ring, 2 rings, or 3 rings. This embodiment does not limit the arrangement of disinfection lamps 10 in the shape of the point. For example, the disinfection lamps 10 can be randomly arranged within a preset range, arranged horizontally, vertically, or in a preset shape. This embodiment does not limit the size of the preset range, for example, it can be half of the setting surface in the terminal, or one-third of the setting surface in the terminal.

The following describes a disinfection method performed by a mobile terminal provided in embodiments of the present disclosure. The disinfection method and the mobile terminal capable of conveniently disinfecting an outside described in the previous text can be referenced and corresponded to each other.

### Embodiment 9

FIG. 10 is a schematic diagram illustrating a state in which a terminal is held with a left hand according to an embodiment of the present disclosure.

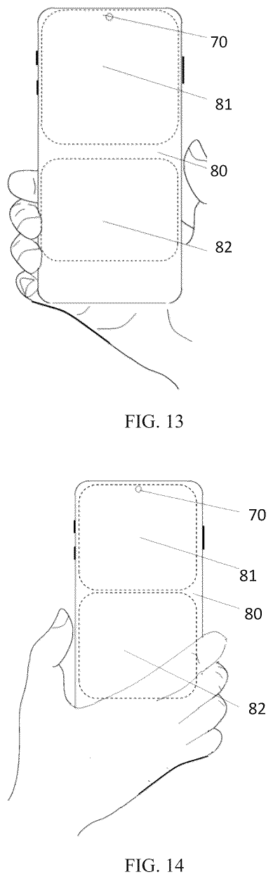

To improve safety and prevent human hands from being accidentally exposed to ultraviolet radiation, it is necessary to limit the way mobile phones are held. This embodiment provides a safe grip mode for left and right hand operation. By providing the front or rear camera image display area 81 and the safe use area in the screen column, the safe use area is the safe use reminder and operation area 82. The safe use reminder and operation area 82 will display the correct grip posture for manual operation during startup, as shown in FIGS. 10 to 14.

In order to further improve safety and prevent human hands from accidentally touching the ultraviolet lamp, turning on the ultraviolet lamp can cause human hands to be exposed to ultraviolet radiation. As shown in FIG. 10, this embodiment provides a schematic diagram of a terminal with a position sensing sensor for left-hand grip. When holding a mobile phone with human hands, the position sensing sensor or the pressure sensor is provided on the left and right sides wall areas at the bottom of the phone, and a left side wall finger sensing area 91 and a right side wall finger sensing area 92 are formed.

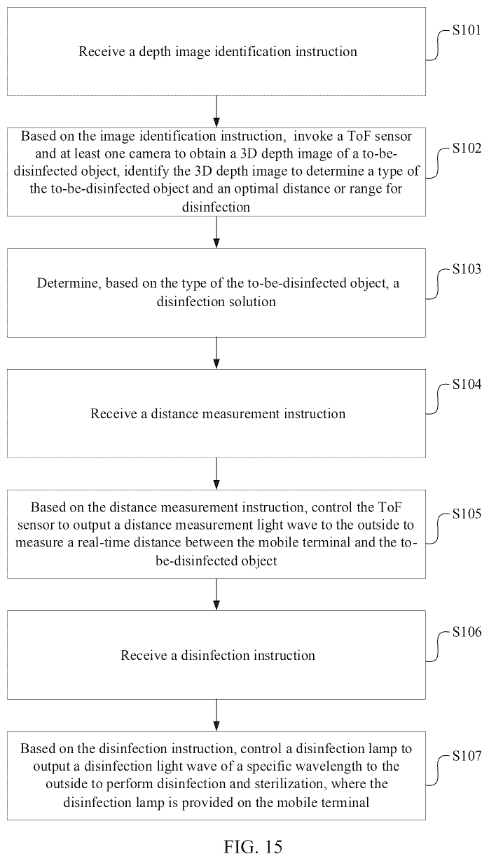

Based on the correct safe grip posture and referring to FIG. 15, the present invention also provides a disinfection method, performed by a mobile terminal, the disinfection method may include the following steps S101 to S107.

At step S101, a depth image identification instruction is received.

The execution subject of this embodiment is a mobile terminal. This embodiment does not limit the type of the mobile terminal as long as it is capable of performing the disinfection method. For example, the mobile terminal could be a mobile phone, or a smart watch, or a smart bracelet, or other wearable smart device, or a satellite communication terminal. The present embodiment does not limit the manner in which the depth image identification instruction can be received, as long as the depth image identification instruction can be received. For example, voice commands may be received, as well as commands entered by the port. This embodiment does not limit the specific content of the depth image identification instruction. For example, it can be an instruction to trigger the start of depth image identification or an instruction to perform a depth image identification operation once. This embodiment does not limit the specific way of port input. For example, port input can be done through hardware keys or shortcut keys, or through APP program instructions.

At step S102, based on the depth image identification instruction, a ToF sensor and at least one camera are invoked to obtain a 3D depth image of a to-be-disinfected object, the 3D depth image is identified to determine a type of the to-be-disinfected object and an optimal distance or range for disinfection.

This embodiment does not limit the basis for determining the optimal disinfection distance or range. For example, the optimal disinfection distance or range can be determined based on the material of the to-be-disinfected object, or based on the intended use of the to-be-disinfected object.

At step S103, based on the type of the to-be-disinfected object, a disinfection solution is determined.

The present embodiment does not limit the manner in which a disinfection solution is determined based on the type of the to-be-disinfected object, as long as it can achieve the disinfection operation on the to-be-disinfected object. For example, a mapping relationship table can be established based on the type of object and the disinfection solution, and the disinfection solution can be determined based on the mapping relationship table between the type of object and the disinfection solution.

At step S104, a distance measurement instruction is received.

The present embodiment does not limit the manner in which the distance measurement instruction can be received, as long as the distance measurement instruction can be received. For example, voice commands may be received, as well as commands entered by the port. This embodiment does not limit the specific content of the distance measurement instruction. For example, it can be an instruction to trigger the start of distance measurement or an instruction to perform a distance measurement operation once. This embodiment does not limit the preset conditions for triggering the program to receive the distance measurement instruction. For example, it can be a program that receives a distance measurement instruction every time a to-be-disinfected object is disinfected, that is, a program that receives a distance measurement instruction is executed every time a disinfection instruction is received, or it can be a program that receives a distance measurement instruction after receiving an instruction to enable distance measurement. This embodiment does not limit the triggering sequence of programs that receive the disinfection instruction and the distance measurement instruction. It can be a program that triggers both the distance measurement instruction and the disinfection instruction simultaneously, or a program that triggers the disinfection instruction after receiving the distance measurement instruction, or a program that triggers the distance measurement instruction after receiving the disinfection instruction. This embodiment does not limit the frequency of receiving the distance measurement instruction. For example, real-time reception can be performed, or reception can be performed every preset reception time. This embodiment does not limit the set value of the preset reception time. For example, it can be 1 second or 2 seconds.

At step S105, based on the distance measurement instruction, the ToF sensor is controlled to output a distance measurement light wave to the outside to measure a real-time distance between the mobile terminal and the to-be-disinfected object.

At step S106, a disinfection instruction is received.

The present embodiment does not limit the manner in which the disinfection instruction can be received, as long as the distance disinfection instruction can be received. For example, voice commands may be received, as well as commands entered by the port. This embodiment does not limit the specific content of the disinfection instruction. For example, it can be an instruction to trigger the start of disinfection, an instruction to perform a disinfection operation, or an instruction containing disinfection parameters. This embodiment does not limit the specific content of the instruction containing disinfection parameters. For example, it can be the instruction containing the type of disinfection light wave or the instruction containing the corresponding number of target disinfection lamps. This embodiment does not limit the number of corresponding target disinfection lamps, for example, it can be 1 or 3. This embodiment does not limit the duration of executing a disinfection instruction once, for example, it can be 2 minutes, 5 minutes, or 10 minutes. This embodiment does not limit the specific way of port input. For example, port input can be done through hardware keys or shortcut keys, or through APP program instructions.

At step S107, based on the disinfection instruction, a disinfection lamp is controlled to output a disinfection light wave of a specific wavelength to the outside to perform disinfection and sterilization, where the disinfection lamp is provided on the mobile terminal.

It is to be noted that in this embodiment, the disinfection lamp can be controlled to output disinfection light waves of specific wavelength through corresponding modules to implement disinfection and sterilization. For example, it can be implemented through a controller. In this embodiment, the controller is connected to the disinfection lamp through one end of the circuit, and is connected to the ToF sensor through the other end of the circuit to control the disinfection lamp to output disinfection light waves and control the ToF sensor to receive disinfection light waves.

This embodiment is not limited to the specific way of providing the disinfection lamp on the mobile terminal. For example, a disinfection lamp can be added on the mobile terminal, or the flash lamp can be integrated with the disinfection lamp, and the disinfection lens can be integrated into the ToF sensor.

This embodiment does not limit the specific location of the disinfection lamp, as long as it can output disinfection light waves of a specific wavelength to the outside. For example, the disinfection lamp may be provided at the back of the mobile terminal, at the front of the mobile terminal, or at the side of the mobile terminal. This embodiment does not limit the number of disinfection lamps, for example, the number of disinfection lamps can be 1, 3, or 4, as long as the disinfection lamp can output disinfection light waves of a specific wavelength to the outside. It is foreseeable that the higher the number of disinfection lamps, the higher the disinfection efficiency.

### Embodiment 10

At step S101, an image identification instruction is received.

The execution subject of this embodiment is a mobile terminal. This embodiment does not limit the type of the mobile terminal as long as it is capable of performing the disinfection method. For example, the mobile terminal could be a mobile phone, or a smart watch, or a smart bracelet, or other wearable smart device, or a satellite communication terminal. The present embodiment does not limit the manner in which the depth image identification instruction can be received, as long as the depth image identification instruction can be received. For example, voice commands may be received, as well as commands entered by the port. This embodiment does not limit the specific content of the depth image identification instruction. For example, it can be an instruction to trigger the start of depth image identification or an instruction to perform a depth image identification operation once. This embodiment does not limit the specific way of port input. For example, port input can be done through hardware keys or shortcut keys, or through APP program instructions.

At step S102, based on the image identification instruction, a time-of-flight (ToF) sensor and at least one camera are invoked to obtain a three-dimensional (3D) depth image of a to-be-disinfected object, the 3D depth image is identified to determine a type of the to-be-disinfected object and an optimal distance or range for disinfection.

This embodiment does not limit the basis for determining the optimal disinfection distance or range. For example, the optimal disinfection distance or range can be determined based on the material of the to-be-disinfected object, or based on the intended use of the to-be-disinfected object.

At step S103, based on the type of the to-be-disinfected object, a disinfection solution is determined.

The present embodiment does not limit the manner in which a disinfection solution is determined based on the type of the to-be-disinfected object, as long as it can achieve the disinfection operation on the to-be-disinfected object. For example, a mapping relationship table can be established based on the type of object and the disinfection solution, and the disinfection solution can be determined based on the mapping relationship table between the type of object and the disinfection solution.

At step S104, a distance measurement instruction is received.

The present embodiment does not limit the manner in which the distance measurement instruction can be received, as long as the distance measurement instruction can be received. For example, voice commands may be received, as well as commands entered by the port. This embodiment does not limit the specific content of the distance measurement instruction. For example, it can be an instruction to trigger the start of distance measurement or an instruction to perform a distance measurement operation once. This embodiment does not limit the preset conditions for triggering the program to receive the distance measurement instruction. For example, it can be a program that receives a distance measurement instruction every time a to-be-disinfected object is disinfected, that is, a program that receives a distance measurement instruction is executed every time a disinfection instruction is received, or it can be a program that receives a distance measurement instruction after receiving an instruction to enable distance measurement. This embodiment does not limit the triggering sequence of programs that receive the disinfection instruction and the distance measurement instruction. It can be a program that triggers both the distance measurement instruction and the disinfection instruction simultaneously, or a program that triggers the disinfection instruction after receiving the distance measurement instruction, or a program that triggers the distance measurement instruction after receiving the disinfection instruction. This embodiment does not limit the frequency of receiving the distance measurement instruction. For example, real-time reception can be performed, or reception can be performed every preset reception time. This embodiment does not limit the set value of the preset reception time. For example, it can be 1 second or 2 seconds.

At step S105, based on the distance measurement instruction, the ToF sensor is controlled to output a distance measurement light wave to the outside to measure a real-time distance between the mobile terminal and the to-be-disinfected object.

At step S106, a disinfection instruction is received.

The present embodiment does not limit the manner in which the disinfection instruction can be received, as long as the distance disinfection instruction can be received. For example, voice commands may be received, as well as commands entered by the port. This embodiment does not limit the specific content of the disinfection instruction. For example, it can be an instruction to trigger the start of disinfection, an instruction to perform a disinfection operation, or an instruction containing disinfection parameters. This embodiment does not limit the specific content of the instruction containing disinfection parameters. For example, it can be the instruction containing the type of disinfection light wave or the instruction containing the corresponding number of target disinfection lamps. This embodiment does not limit the number of corresponding target disinfection lamps, for example, it can be 1 or 3. This embodiment does not limit the duration of executing a disinfection instruction once, for example, it can be 2 minutes, 5 minutes, or 10 minutes. This embodiment does not limit the specific way of port input. For example, port input can be done through hardware keys or shortcut keys, or through APP program instructions.

At step S107, based on the disinfection instruction, a disinfection lamp is controlled to output a disinfection light wave of a specific wavelength to an outside to perform disinfection and sterilization, where the disinfection lamp is provided on the mobile terminal.

It is to be noted that in this embodiment, the disinfection lamp can be controlled to output disinfection light waves of specific wavelength through corresponding modules to implement disinfection and sterilization. For example, it can be implemented through a controller. In this embodiment, the controller is connected to the disinfection lamp through one end of the circuit, and is connected to the ToF sensor through the other end of the circuit to control the disinfection lamp to output disinfection light waves and control the ToF sensor to receive disinfection light waves.

This embodiment is not limited to the specific way of providing the disinfection lamp on the mobile terminal. For example, a disinfection lamp can be added on the mobile terminal, or the flash lamp can be integrated with the disinfection lamp, and the disinfection lens can be integrated into the ToF sensor.

This embodiment does not limit the specific location of the disinfection lamp, as long as it can output disinfection light waves of a specific wavelength to the outside. For example, the disinfection lamp may be provided at the back of the mobile terminal, at the front of the mobile terminal, or at the side of the mobile terminal. This embodiment does not limit the number of disinfection lamps, for example, the number of disinfection lamps can be 1, 3, or 4, as long as the disinfection lamp can output disinfection light waves of a specific wavelength to the outside. It is foreseeable that the higher the number of disinfection lamps, the higher the disinfection efficiency.

Further, in order to accurately disinfect and sterilize different to-be-disinfected objects and improve the disinfection effect of the disinfection lamp, the disinfection solution can include at least one of: a disinfection light wave parameter, a disinfection start time, or a duration.

In this embodiment, the disinfection parameter can be a light wave parameter, a disinfection start time, or a duration. This embodiment does not limit the content of disinfection parameters, for example, it can be one of the above-mentioned disinfection parameters, or any combination of the above-mentioned disinfection parameters. It is be noted that there is a corresponding relationship between the disinfection parameter and the type of to-be-disinfected object, for example, it can be associated in the form of a mapping table, where the mapping table is a preset mapping relationship. This embodiment does not limit the content of the mapping relationship. For example, the light wave parameter and the disinfection start time can be used as a first disinfection parameter, corresponding to the first type of to-be-disinfected object. The light wave parameter and the duration can also be used as a second disinfection parameter, corresponding to the second type of to-be-disinfected object. The light wave parameter, the disinfection start time, and the duration can also be used as a third disinfection parameter, corresponding to the third type of to-be-disinfected object.

Further, in order to output the corresponding target disinfection light wave according to the disinfection instruction and improve the disinfection effect of the disinfection lamp, the disinfection lamp can be controlled to output the disinfection light wave of a specific wavelength according to the disinfection instruction to implement disinfection and sterilization, which can include:
determining the target disinfection light wave of the disinfection lamp according to the disinfection instruction; and
controlling the disinfection lamp to output the target disinfection light wave to implement disinfection and sterilization.

The type of target disinfection light wave in this embodiment is determined according to the disinfection instruction, and the type of light wave determined according to the disinfection instruction can be ultraviolet light, femtosecond laser, blue light, or near-infrared light. In this embodiment, the disinfection lamp is controlled to output the target disinfection light wave to implement disinfection and sterilization. It is to be noted that this embodiment does not limit the duration of disinfection and sterilization by outputting the target disinfection light wave, for example, it can be 1 minute or 3 minutes.

Furthermore, in order to avoid being damaged by disinfection light waves during use, according to the depth image recognition instructions, the method further includes safe use confirmation including:
confirming, by invoking a front camera, that a disinfection user is an authenticated and authorized user, and determining whether a current state is a safe use state; and
in response to determining that the current state is the safe use state, starting a depth image identification process.

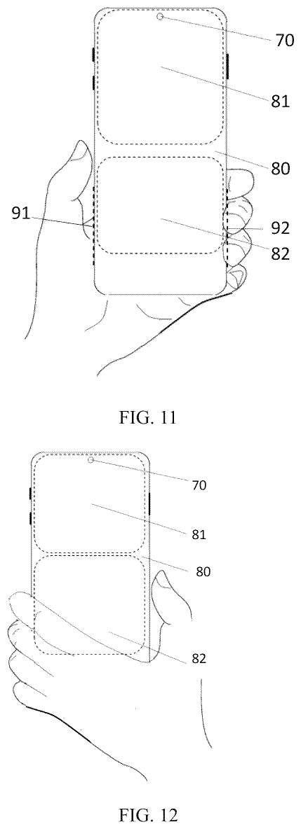

This embodiment does not limit the way of determining whether the current state is a safe state. For example, it can be determined by providing a pressure sensor to detect the grip position, as shown in FIG. 11. It can also be determined by detecting the grip position through a temperature sensor, or by manually judging the grip position to determine whether it is in a safe state.

Furthermore, in order to reduce the cost of the mobile terminal, determining whether the current state is the safe use state further includes:
displaying a safe use area for holding the mobile terminal on a display screen of the mobile terminal, where the safe use area refers to the safe use reminder and operation area 82, as shown in FIGS. 10 to 14.

It is determined, by the safe use area displayed, whether a palm or fingers of a current user operating the mobile terminal is in the safe use state.

This embodiment does not limit the display area of the safe use area in the display screen, as long as it can output and display the safe use area. For example, it can be half of the total area of the display screen, or one-third of the total area of the display screen. This embodiment does not limit the specific content of displaying the safe use area, for example, it can be a schematic diagram of the hand grip area or a schematic diagram of the placement area of the mobile terminal. This embodiment does not limit the specific way of determining the current safe use state, for example, it can be confirmed by the user clicking or automatically confirmed by the mobile terminal. This embodiment does not limit the automatic confirmation method of the mobile terminal, for example, confirmation can be based on the pressure sensor or the temperature sensor. This embodiment does not limit the specific content of the schematic diagram. For example, it can be a schematic diagram of the terminal position marked with the placement area, or a schematic diagram marked with the position of the warning line. As long as it can output the prompt information of the safe use area, it is sufficient. The safe use area is as shown in FIGS. 10 to 14.

Furthermore, to improve the accuracy of confirming the safe use state, the safe use state represents that a position of the palm or the fingers of the current user is sensed by a position sensing sensor provided on a surface of the mobile terminal to confirm that the palm or the fingers is in a safe use position, and the position sensing sensor is connected to the controller, as shown in FIG. 11.

Further, in order to ensure that the to-be-disinfected object is within the effective area of the disinfection lamp, and to improve the disinfection efficiency of the disinfection lamp, controlling the disinfection lamp to output the disinfection light wave of the specific wavelength to the outside to perform disinfection and sterilization may include:
determining whether the to-be-disinfected object is in the optimal distance or range of the disinfection lamp; and
in response to the to-be-disinfected object being in the optimal disinfection distance range of the disinfection lamp, controlling the disinfection lamp to output the disinfection light wave of the specific wavelength to the outside to perform disinfection and sterilization.

This embodiment does not limit the specific mode of determining whether the to-be-disinfected object is within the optimal disinfection distance range of the disinfection lamp. For example, it can be determined by the distance between the disinfection lamp and the to-be-disinfected object. If the distance between the disinfection lamp and the to-be-disinfected object is less than the preset distance threshold, it can be confirmed that the to-be-disinfected object is within the optimal disinfection distance range of the disinfection lamp. It may also be determined by the percentage of the area of the to-be-disinfected object to the area of the effective area in the disinfection lamp. If the percentage of the area of the to-be-disinfected object to the area of the effective area in the disinfection lamp is greater than a preset percentage threshold, it is confirmed that the to-be-disinfected object is within the optimal disinfection distance range of the disinfection lamp. It can also be determined by combining the above-mentioned two modes. Accordingly, this embodiment can use a camera to obtain the percentage of the area of the to-be-disinfected object to the area of the effective area in the disinfection lamp, to determine whether the to-be-disinfected object is within the optimal disinfection distance range of the disinfection lamp. It may also be determined whether the to-be-disinfected object is within the optimal disinfection distance range of the disinfection lamp by obtaining the distance between the to-be-disinfected object and the disinfection lamp through a distance sensor. It is to be noted that if the to-be-disinfected object is in the optimal disinfection distance range of the disinfection lamp, controlling the disinfection lamp to output the disinfection light wave of the specific wavelength to the outside to perform disinfection and sterilization.

Furthermore, in order to reduce the cost of the mobile terminal and improve the accuracy of determining whether the to-be-disinfected object is within the optimal disinfection distance range of the disinfection lamp, determining whether the to-be-disinfected object is within the optimal disinfection distance range of the disinfection lamp may include:
displaying, by invoking the camera, the optimal disinfection distance range of the disinfection lamp and a real-time video image of the to-be-disinfected object on the display screen of the mobile terminal; and
determining whether the to-be-disinfected object is within the optimal disinfection distance range of the disinfection lamp by using the displayed optimal disinfection distance range of the disinfection lamp.

In this embodiment, the camera is called to display the optimal disinfection distance range of the disinfection lamp on the display screen of the mobile terminal, and the optimal disinfection distance range of the disinfection lamp displayed on the display screen is used to determine whether the to-be-disinfected object is within the optimal disinfection distance range of the disinfection lamp. It is to be noted that this embodiment does not limit the specific mode of determining whether the to-be-disinfected object is within the optimal disinfection distance range of the disinfection lamp. For example, it can be manually determined, or it can be automatically judged by the mobile terminal. The mobile terminal can determine based on the area proportion of the to-be-disinfected object within the optimal disinfection distance range of the disinfection lamp. When the area proportion is greater than a preset threshold, it can be determined that the to-be-disinfected object is within the optimal disinfection distance range of the disinfection lamp. This embodiment does not limit the set value of the preset threshold, for example, it can be 50% or 70%. This embodiment does not limit the basis for setting the preset threshold value. For example, it can be set based on the size of the to-be-disinfected object, and the larger the volume of the to-be-disinfected object, the larger the preset threshold value.

It can be seen that the disinfection method of the mobile terminal provided by the embodiment of the present disclosure can easily implement the disinfection and sterilization of the to-be-disinfected object by adding a disinfection lamp on the mobile terminal and equipping it with a controller. According to the received depth image recognition command, the ToF sensor and at least one camera are called according to the depth image identification instruction to obtain the 3D depth image of the to-be-disinfected object, and the image is identified to determine the type of object and the optimal disinfection distance or range, and the disinfection solution is determined according to the type of object. Different disinfection solutions can be implemented for different objects to be disinfected, so as to improve the disinfection efficiency. A distance measurement instruction is received, according to the distance measurement instruction, the corresponding ToF sensor is controlled to output the distance measurement light wave, so as to measure the real-time distance between the mobile terminal and the to-be-disinfected object, and obtain the real-time distance between the to-be-disinfected object and the mobile terminal in real time. According to the disinfection instruction, the disinfection lamp is controlled to output the target disinfection light wave to implement disinfection and sterilization, so as to improve the disinfection efficiency of the disinfection lamp. By calling the camera to determine the type of to-be-disinfected object, and then determine the corresponding disinfection parameters, the corresponding disinfection lamp is controlled to implement disinfection and sterilization according to the disinfection parameters, and can accurately disinfect different to-be-disinfected objects, and improve the disinfection effect of the disinfection lamp. Determining whether the mobile terminal is currently in a safe use state, and determining whether the current to-be-disinfected object is within the optimal disinfection distance range of the disinfection lamp and making adjustments, can avoid causing harm to the user, ensure the effect of disinfecting the to-be-disinfected object, improve the effect of disinfecting viruses or bacteria, and thus reduce the risk caused by the spread of viruses and bacteria.

### Embodiment 11

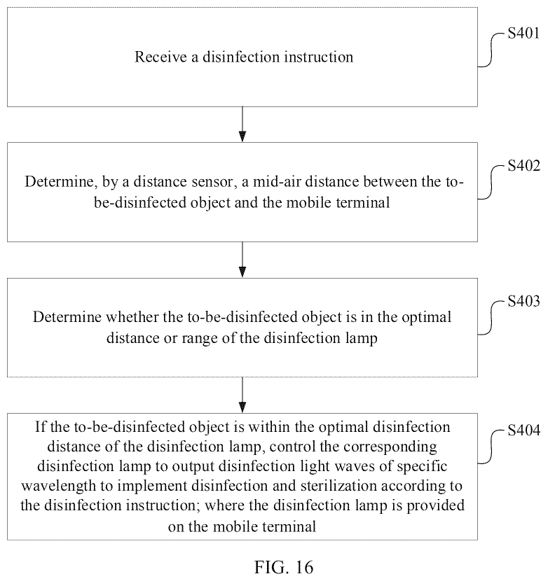

FIG. 16 is a flowchart illustrating a disinfection method of a mobile terminal according to another embodiment of the present disclosure. As shown in FIG. 16, the disinfection method may include the following steps S401 to S404.

At step S401, a disinfection instruction is received.

At step S402, a mid-air distance between the to-be-disinfected object and the mobile terminal is determined by a distance sensor.

At step S403, it is determined whether the to-be-disinfected object is in the optimal distance or range of the disinfection lamp.

It is to be noted that the distance sensor is used to determine a mid-air distance between the to-be-disinfected object and the mobile terminal. When the mid-air distance is less than or equal to a preset distance threshold, it is determined that the to-be-disinfected object is within the optimal disinfection distance range of the disinfection lamp. This embodiment does not limit the basis for setting the preset distance threshold. For example, it can be set based on the wavelength of the disinfection light wave output by the disinfection lamp in the mobile terminal, or it can be set based on the type of disinfection light wave output by the disinfection lamp in the mobile terminal. This embodiment does not limit the type of distance sensor. For example, it can be an infrared light sensor, a ToF sensor, or an RGBD depth sensor.

At step S404, if the to-be-disinfected object is within the optimal disinfection distance of the disinfection lamp, the corresponding disinfection lamp is controlled to output disinfection light waves of specific wavelength to implement disinfection and sterilization according to the disinfection instruction; where the disinfection lamp is provided on the mobile terminal.

Furthermore, in order to improve the accuracy of determining the optimal disinfection distance range, the above-mentioned determination of whether the to-be-disinfected object is within the optimal disinfection distance range of the disinfection lamp also includes invoking a camera to obtain a real-time image, comparing the real-time image with the 3D depth image of the to-be-disinfected object, and identifying whether an abnormal object suddenly appears. If the abnormal object is identified, the disinfection lamp stops outputting disinfection light waves, where the abnormal object includes other people's palms or fingers.

Furthermore, in order to provide timely feedback on situations where the to-be-disinfected object is not within the optimal disinfection distance range of the disinfection lamp, when the to-be-disinfected object is not within the optimal disinfection distance range of the disinfection lamp, the disinfection method may include:
in response to the to-be-disinfected object being not in the effective area of the disinfection lamp, outputting adjustment prompt information.

This embodiment does not limit the way of outputting the adjustment prompt information, as long as the adjustment prompt information can provide feedback in a timely manner when the to-be-disinfected object is not within the optimal disinfection distance range of the disinfection lamp. For example, the adjustment prompt information can be output in the form of sound, image or text, or a combination thereof. This embodiment is not limited to the specific content output in the form of sound. For example, it can be adjusted through voice prompts, alarms, or output adjustments, such as approaching or moving away from the voice output. Correspondingly, this embodiment is not limited to outputting specific content in the form of images or text, for example, it can be outputting warning signs or red exclamation marks. It can also be output in the form of scrolling text. It is to be noted that based on the output adjustment prompt information, the position of the to-be-disinfected object and the position of the mobile terminal can be adjusted, so that the to-be-disinfected object is within the optimal disinfection distance range of the disinfection lamp.

Furthermore, in order to output real-time disinfection information and improve user experience, the disinfection method may also include:
indicating a progress of disinfection.

This embodiment does not limit the specific way of indicating the progress of disinfection, for example, it can be indicated at the beginning of disinfection, at 50% completion of disinfection, or at 100% completion of disinfection. This embodiment does not limit the frequency of prompting the progress of disinfection, for example, it can be done once every preset prompt time period, or it can only prompt a preset number of times. This embodiment does not limit the set value of the preset number of times, it can be 2 times or 3 times. Correspondingly, this embodiment does not limit the set value of the preset prompt time period, for example, it can be 5 minutes or 8 minutes. This embodiment does not limit the specific content of indicating the progress of disinfection, which can be indicating the remaining disinfection time or the percentage of completed disinfection. This embodiment does not limit the specific content of indicating the progress of disinfection, which can be indicating the remaining disinfection time or the percentage of completed disinfection. This embodiment does not limit the specific way of indicating the progress of disinfection. For example, it can be indicated through voice or screen display. This embodiment does not limit the specific manifestation of the prompt displayed on the screen, for example, the prompt can be displayed in the form of text or progress bars.

Furthermore, in order to improve the accuracy of the distance measurement for the to-be-measured object, controlling the ToF sensor to calculate the distance between the mobile terminal and the to-be-measured object based on the disinfection light wave may include:
controlling the laser emitter of the ToF sensor to output distance measurement laser; and
controlling the ToF processor to control the ToF sensor to calculate the distance between the mobile terminal and the to-be-measured object based on the distance measurement laser and the disinfection light wave reflected by the to-be-measured object.

It is to be noted that this embodiment can control the ToF processor of the ToF sensor through the controller, and then control the laser emitter to output the distance measurement laser through the ToF processor. In this embodiment, one end of the ToF processor is connected to the controller, and the other end of the ToF processor is connected to the laser emitter end and the laser receiver. The disinfection method of the mobile terminal provided by the embodiment of the present disclosure conveniently disinfects the to-be-disinfected object by adding a disinfection lamp on the mobile terminal and providing a controller on the mobile terminal. According to the disinfection instruction, the disinfection lamp is controlled to output the target disinfection light wave to implement disinfection and sterilization, such that the disinfection efficiency of the disinfection lamp is improved. It is identified whether an abnormal object emerges by calling the camera to obtain a real-time image and comparing the real-time image with the 3D depth image of the to-be-disinfected object. If an abnormal object is identified, the disinfection lamp stops outputting the disinfection light wave, where the abnormal object includes other people's palms or fingers. It is determined whether the to-be-disinfected object is within the optimal disinfection distance range of the disinfection lamp, such that the accuracy of determining the optimal disinfection distance range is improved. The distance between the mobile terminal and the to-be-disinfected object is determined by using the distance measurement laser and the disinfection light wave through the ToF sensor, such that the accuracy of distance measurement is improved. By adding a distance sensor, the distance between the mobile terminal and the to-be-disinfected object can be measured in a timely manner, which ensures the disinfection efficiency of the disinfection lamp, improves the disinfection and sterilization effect of viruses or bacteria, thus reducing the risk caused by the spread of viruses and bacteria. By prompting the progress of disinfection, the user experience of mobile terminals is improved.

In order to make this disclosure easier to understand, this embodiment takes a mobile phone as an example for explanation, which can specifically include the following steps S1 to S10.

At step S1, the disinfection application is launched to trigger the disinfection instruction.

The disinfection application of the mobile phone is launched to trigger the disinfection instruction.

At step S2, the camera is invoked to display the effective irradiation area of the disinfection light.

The mobile terminal invokes the camera to display the effective disinfection light irradiation area, and determines that the to-be-disinfected object is within the effective disinfection light irradiation area.

At step S3, a safe area to hold the mobile terminal is displayed.

The mobile terminal displays the safe area to hold the mobile terminal to determine whether the mobile terminal is currently in a safe use state.

At step S4, If the safe use test is qualified, the distance between the mobile phone and the to-be-disinfected object is checked.

At step S5, it is determined whether the distance between the mobile terminal and the to-be-disinfected object is within an effective distance according to the prompt. If the distance between the mobile terminal and the to-be-disinfected object is within an effective distance, the step S6 is performed.

At step S6, irradiation disinfection is launched and the progress of disinfection is displayed.

The phone shows the progress of disinfection in the form of a progress bar through the display screen.

At step S7, the disinfection operation is completed, the disinfection light irradiation is automatically stopped, and the completion of the operation is prompted.

Furthermore, the steps may also include:
At step S8, a distance measurement instruction is received.

At step S9, a corresponding disinfection lamp is controlled to output a specific wavelength of disinfection light wave to the outside, and the laser emitter of the ToF sensor is controlled to output a distance measurement laser.

At step S10, the ToF processor is controlled to control the ToF sensor to calculate the distance between the mobile terminal and the to-be-measured object based on the distance measurement laser and the disinfection light wave reflected by the to-be-measured object.

The following describes a disinfection apparatus of a mobile terminal provided in embodiments of the present disclosure. The disinfection apparatus and the disinfection method of the mobile terminal capable of conveniently disinfecting an outside described in the previous text can be referenced and corresponded to each other.

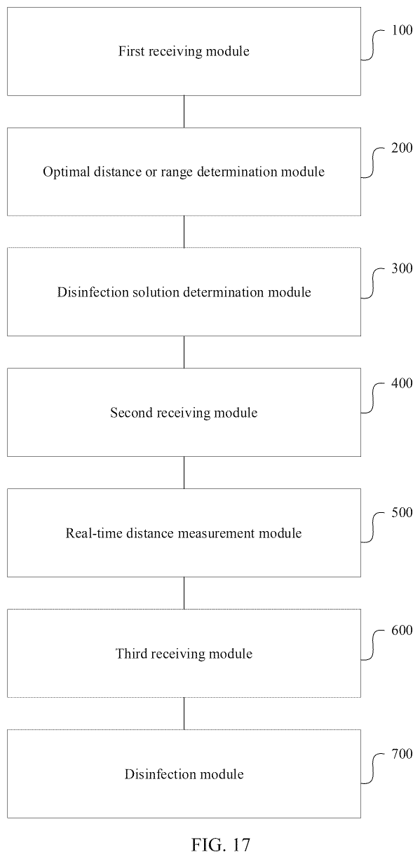

FIG. 17 is a block diagram illustrating a disinfection apparatus of a mobile terminal according to another embodiment of the present disclosure. The disinfection apparatus includes a first receiving module 100, an optimal distance or range determination module 200, a disinfection solution determination module 300, a second receiving module 400, a real-time distance measurement module 500, a third receiving module 600, and a disinfection module 700.

The first receiving module 100 is configured to receive an image identification instruction.

The optimal distance or range determination module 200 is configured to invoke, based on the image identification instruction, a ToF sensor and at least one rear camera to obtain a three-dimensional (3D) depth image of a to-be-disinfected object, and identify the 3D depth image to determine a type of the to-be-disinfected object and an optimal distance or range for disinfection.

The disinfection solution determination module 300 is configured to determine, based on the type of the to-be-disinfected object, a disinfection solution.

The second receiving module 400 is configured to receive a distance measurement instruction.

The real-time distance measurement module 500 is configured to control, based on the distance measurement instruction, the ToF sensor to output a distance measurement light wave to the outside to measure a real-time distance between the mobile terminal and the to-be-disinfected object.

The third receiving module 600 is configured to receive a disinfection instruction.

The disinfection module 700 is configured to control, based on the disinfection instruction, a disinfection lamp to output a disinfection light wave of a specific wavelength to an outside to perform disinfection and sterilization, where the disinfection lamp is provided on the mobile terminal.

Based on the embodiments, the disinfection solution determined by the disinfection solution determination module 300 can include at least one of: a disinfection light wave parameter, a disinfection start time, or a duration.

Based on any of the embodiments, the disinfection apparatus of the mobile terminal may further include a safe use determination module, which may include: a safe use state determination unit and a deep image identification process activation unit.

The safe use state determination unit is configured to determine, by invoking a front camera, that a disinfection user is an authenticated and authorized user, and determine whether a current state is a safe use state.

The deep image identification process activation unit is configured to: in response to determining that the current state is the safe use state, start a depth image identification process.

Based on any of the embodiments, the safe use state determination unit may further include: a display subunit and a safe use state determination subunit.

The display subunit is configured to display a safe use area for holding the mobile terminal on a display screen of the mobile terminal.

The safe use state determination subunit is configured to determine, by the safe use area displayed, whether a palm or fingers of a current user operating the mobile terminal is in the safe use state.

Based on the embodiments, to improve the accuracy of confirming the safe use state, the safe use state determined by the safe use state determination unit represents that a position of the palm or the fingers of the current user is sensed by a position sensing sensor provided on a surface of the mobile terminal to confirm that the palm or the fingers is in a safe use position, and the position sensing sensor is connected to the controller.

Based on the embodiments, the disinfection module 700 may include: an optimal distance or range determination unit and a first performing unit.

The optimal distance or range determination unit is configured to determine whether the to-be-disinfected object is within the optimal disinfection distance range of the disinfection lamp.

The first performing unit is configured to: if the to-be-disinfected object is in the optimal disinfection distance range of the disinfection lamp, control the disinfection lamp to output the disinfection light wave of the specific wavelength to the outside to perform disinfection and sterilization.

Based on the embodiments, the optimal distance or range determination unit may include: a camera invoking subunit and a first optimal distance or range determination subunit.

The camera invoking subunit is configured to invoke the camera to display the optimal disinfection distance range of the disinfection lamp and a real-time video image of the to-be-disinfected object on the display screen of the mobile terminal.

The first optimal distance or range determination subunit is configured to determine whether the to-be-disinfected object is within the optimal disinfection distance range of the disinfection lamp by using the displayed optimal disinfection distance range of the disinfection lamp.

Based on the embodiments, the optimal distance or range determination subunit may include: a mid-air distance determination subunit and a second optimal distance or range determination subunit.

The mid-air distance determination subunit is configured to determine, by a distance sensor, a mid-air distance between the to-be-disinfected object and the mobile terminal.

The second optimal distance or range determination subunit is configured to determine whether the to-be-disinfected object is within the optimal disinfection distance range of the disinfection lamp by using the mid-air distance.

Based on the embodiments, the second optimal distance or range determination subunit is further configured to invoke a camera to obtain a real-time image, compare the real-time image with the 3D depth image of the to-be-disinfected object, and identify whether an abnormal object suddenly appears. If the abnormal object is identified, the disinfection lamp stops outputting disinfection light waves, where the abnormal object includes other people's palms or fingers.

Based on the embodiments, the second optimal distance or range determination subunit may include: a prompt subunit.

The prompt subunit is configured to output adjustment prompt information.

Based on the embodiments, the disinfection apparatus of the mobile terminal may also include: an indication module.

The indication module is configured to indicate a progress of disinfection.

In the disinfection apparatus of the mobile terminal, the first receiving module 100 is configured to receive an image identification instruction; the optimal distance or range determination module 200 is configured to invoke, based on the image identification instruction, a ToF sensor and at least one rear camera to obtain a 3D depth image of a to-be-disinfected object, identify the 3D depth image to determine a type of the to-be-disinfected object and an optimal distance or range for disinfection; the disinfection solution determination module 300 is configured to determine, based on the type of the to-be-disinfected object, a disinfection solution; the second receiving module 400 is configured to receive a distance measurement instruction; the real-time distance measurement module 500 is configured to control, based on the distance measurement instruction, the ToF sensor to output a distance measurement light wave to the outside to measure a real-time distance between the mobile terminal and the to-be-disinfected object; the third receiving module 600 is configured to receive a disinfection instruction; and the disinfection module 700 is configured to control, based on the disinfection instruction, a disinfection lamp to output a disinfection light wave of a specific wavelength to an outside to perform disinfection and sterilization, where the disinfection lamp is provided on the mobile terminal. By adding the disinfection lamp and the ToF sensor on the mobile terminal, and providing a controller, the present disclosure can conveniently disinfect the to-be-disinfected object, and perform the distance measurement on the to-be-measured object, which improves the accuracy of distance measurement of the to-be-measured object. According to the disinfection instruction, the disinfection lamp can output target disinfection light waves to implement disinfection and sterilization, which can output multiple target disinfection light waves at the same time, improve the disinfection efficiency of the disinfection lamp. By invoking the camera to determine the type of to-be-disinfected object, then determine the corresponding disinfection parameters, control the corresponding disinfection lamp to implement disinfection and sterilization according to the disinfection parameters, which can accurately disinfect different to-be-disinfected objects, and improve the disinfection effect of the disinfection lamp. It is determined whether the mobile terminal that can conveniently disinfect the outside is currently in safe use state, and determine whether the to-be-disinfected object is currently within the effective area of the disinfection lamp and make the adjustment to avoid harm to the user, ensure the effect of disinfection and sterilization of the to-be-disinfected object. By adding a distance sensor, the distance between the mobile terminal that can conveniently disinfect the outside and the items to be disinfected can be timely measured, the disinfection efficiency of the disinfection lamp is ensured, the effect of disinfection and sterilization of viruses or bacteria is improved, thereby the risk caused by the spread of viruses and bacteria is reduced, and the user's experience of using the mobile terminals that can conveniently disinfect the outside by prompting the progress of disinfection is improved.

The storage medium provided in embodiments of the present disclosure are described below, and the storage medium and the disinfection method may be referred to in correspondence with each other.

The present disclosure provides a storage medium, storing computer programs thereon, where the computer programs, when executed by a processor, cause the processor to perform the disinfection method of any one of the embodiments.

The above storage media include: a U disk, a mobile hard disk, a read-only memory (ROM), a magnetic disk, or a compact disk and other media that can store program codes.

Accordingly, the mobile terminal disclosed in the present disclosure that can conveniently disinfect to the outside is provided with: the disinfection lamp for outputting the disinfection light wave of a specific wavelength to the outside to disinfect and sterilize a to-be-disinfected object, and the disinfection lamp is combined with the ToF sensor technology, and measures, by means of a 3D depth image, the distance between the to-be-measured object and the mobile terminal and identifies the type of to-be-disinfected object and determines the disinfection solution, such that the ultraviolet disinfection is always carried out at the highest efficiency. On the one hand, the effect of disinfection and sterilization is improved; and on the other hand, the time of use of ultraviolet rays can be effectively reduced and the risk of exposure of the human body to the UV rays is reduced, so that the chances of the ultraviolet rays being harmful to the human body are greatly reduced, and the safety is improved.

The present disclosure further strengthens the safety of use by displaying a safe operation use image and indication on the same screen in addition to a range of effective distance for disinfection in the display screen, and by setting multiple safe use disinfection methods in the disinfection method.

The mobile phone with a disinfection function provided by the present disclosure can reduce the risk caused by the spread of viruses and bacteria, improve the user experience of using a mobile terminal that can conveniently disinfect the outside, and have high economic and social benefits.

The embodiments in this specification are all described in a progressive manner. Descriptions of each embodiment focus on differences from other embodiments, and same or similar parts among respective embodiments may be mutually referenced. As for the apparatus embodiment, since it substantially corresponds to the method embodiment, the description thereof is relatively simple, and relevant parts can be referred to the description of the method embodiment.

The professional may further realize that the units and algorithmic steps of the various examples described in conjunction with the embodiments disclosed herein are capable of being implemented in electronic hardware, computer software, or a combination of both, and that the compositions and steps of the various examples have been described in the foregoing description in general terms according to function in order to clearly illustrate the interchangeability of the hardware and software. Whether a function is performed as hardware or computer software driving hardware depends on the particular application and design constraints of the technical solution. Those skilled in the art can use different methods to realize the described functions for each specific application, but this realization should not be considered beyond the scope of the technical solutions of the embodiments of the present disclosure.

At last, it should be noted that the relational terms herein such as first and second are used only to differentiate an entity or operation from another entity or operation, and do not require or imply any actual relationship or sequence between these entities or operations. Also, the term “including”, “containing” or any variation thereof is intended to encompass non-exclusive inclusion, so that a process, method, article or device including a series of elements includes not only those elements but also other elements not listed explicitly or those elements inherent to such a process, method, article or device.

The mobile terminal capable of conveniently disinfecting the outside, the disinfection method and apparatus of the mobile terminal, and the storage medium of the present disclosure are described above in detail. The principle and implementation of the present disclosure are described herein through specific examples. The description about the embodiments of the present disclosure is merely provided for ease of understanding of the method and core ideas of the present disclosure. Persons of ordinary skill in the art can make variations and modifications to the present disclosure in terms of the specific implementations and application scopes according to the ideas of the present disclosure. Therefore, the specification shall not be construed as a limit to the present disclosure.

1. A mobile terminal capable of conveniently disinfecting an outside, being provided with:
a disinfection lamp, configured to output a disinfection light wave of a specific wavelength to the outside to disinfect and sterilize a to-be-disinfected object;
a time-of-flight (ToF) sensor, configured to receive at least one of the disinfection light wave or a distance measurement light wave reflected by a to-be-measured object to measure a distance between the to-be-measured object and the mobile terminal; and
a controller, connected to and controlling the ToF sensor and the disinfection lamp through a circuit.

2. The mobile terminal of claim 1, wherein the ToF sensor is a LiDAR-type laser radar comprising a laser emitter, a receiver, and a ToF processor.

3. The mobile terminal of claim 2, wherein the disinfection lamp and the ToF sensor are provided independently, the ToF processor is connected to the controller through a first circuit, and the disinfection lamp is connected to the controller through a second circuit.

4. The mobile terminal of claim 2, wherein the disinfection lamp is integrated in the ToF sensor, and the ToF sensor is connected to and controls the disinfection lamp, the laser emitter and the receiver through the ToF processor, respectively.

5. The mobile terminal of claim 2, wherein the disinfection lamp and the laser emitter are combined as a single unit;
wherein a laser generator in the laser emitter is capable of emitting, by pulse modulation, a laser for disinfection with high energy and a laser for distance measurement with low energy, and the laser for disinfection is the disinfection light wave.

6. (canceled)

7. The mobile terminal of claim 1, further comprising a power supply component, a display screen, a front camera, a rear camera and a flash lamp, that are connected to the controller and controlled by the controller.

8. The mobile terminal of claim 7, wherein the flash lamp is integrated in the disinfection lamp, and the controller is connected to and controls the flash lamp and the disinfection lamp through a flash-disinfection processor, respectively.

9. The mobile terminal of claim 7, wherein the controller is connected to a flash-disinfection integrated lamp through a flash-disinfection processor to control the flash-disinfection integrated lamp to emit the disinfection light wave or flash for photography.

10. The mobile terminal of claim 1, wherein the disinfection lamp is configured to output ultraviolet rays or ultraviolet lasers to the outside;
wherein the disinfection lamp is a pulsed ultraviolet laser generating component or a pulsed ultraviolet ray generating component;
wherein the disinfection lamp is disposed in a preset area next to the front camera or the rear camera, and the disinfection lamp in the preset area is provided in a form of a plate, a strip or a ring, or a point;
wherein the mobile terminal is a mobile phone, a smart wearable device, or a satellite communication terminal.

11. (canceled)

12. (canceled)

13. (canceled)

14. A disinfection method, performed by a mobile terminal and comprising:
receiving a disinfection instruction; and
controlling, based on the disinfection instruction, a disinfection lamp to output a disinfection light wave of a specific wavelength to an outside to perform disinfection and sterilization, wherein the disinfection lamp is provided on the mobile terminal.

15. The disinfection method of claim 14, further comprising:
receiving an image identification instruction; and
invoking, based on the image identification instruction, a time-of-flight (ToF) sensor and at least one camera to obtain a three-dimensional (3D) depth image of a to-be-disinfected object, identifying the 3D depth image to determine a type of the to-be-disinfected object and an optimal distance or range for disinfection, and determining, based on the type of the to-be-disinfected object, a disinfection solution; wherein the disinfection solution comprises at least one of: a disinfection light wave parameter, a disinfection start time, or a duration.

16. The disinfection method of claim 15, further comprising:
receiving a distance measurement instruction; and
controlling, based on the distance measurement instruction, the ToF sensor to output a distance measurement light wave to the outside to measure a real-time distance between the mobile terminal and the to-be-disinfected object.

17. (canceled)

18. The disinfection method of claim 15, wherein based on the image identification instruction, the method further comprises safe use confirmation comprising:
confirming, by invoking a front camera, that a disinfection user is an authenticated and authorized user, and determining whether a current state is a safe use state; and
in response to determining that the current state is the safe use state, starting a depth image identification process.

19. The disinfection method of claim 18, wherein determining whether the current state is the safe use state comprises:
displaying a safe use area for holding the mobile terminal on a display screen of the mobile terminal; and
determining, by the safe use area displayed, whether a palm or fingers of a current user operating the mobile terminal is in the safe use state; wherein the safe use state represents that a position of the palm or the fingers of the current user is sensed by a position sensing sensor provided on a surface of the mobile terminal to confirm that the palm or the fingers is in a safe use position, and the position sensing sensor is connected to the controller.

20. (canceled)

21. The disinfection method of claim 14, wherein controlling the disinfection lamp to output the disinfection light wave of the specific wavelength to the outside to perform disinfection and sterilization comprises:
determining whether the to-be-disinfected object is in an effective area of the disinfection lamp; and
in response to the to-be-disinfected object being in the effective area of the disinfection lamp, controlling the disinfection lamp to output the disinfection light wave of the specific wavelength to the outside to perform disinfection and sterilization.

22. The disinfection method of claim 21, wherein determining whether the to-be-disinfected object is in the effective area of the disinfection lamp comprises:
displaying, by invoking a camera, the effective area of the disinfection lamp and a real-time video image of the to-be-disinfected object on the display screen of the mobile terminal, wherein an icon of the effective area is displayed in the real-time video image of the to-be-disinfected object; and
determining, by the effective area of the disinfection lamp displayed, whether the to-be-disinfected object is in the effective area of the disinfection lamp.

23. The disinfection method of claim 22, wherein determining whether the to-be-disinfected object is in the effective area of the disinfection lamp comprises:
determining, by a distance sensor, a mid-air distance between the to-be-disinfected object and the mobile terminal; and
determining, by the mid-air distance, whether the to-be-disinfected object is in the effective area of the disinfection lamp.

24. The disinfection method of claim 23, wherein determining whether the to-be-disinfected object is in the effective area of the disinfection lamp further comprises: invoking the camera to obtain a real-time image, and comparing the real-time image with a 3D depth image of the to-be-disinfected object, identifying whether there is an abnormal object, and in response to there being the abnormal object, the disinfection lamp stops outputting the disinfection light wave, wherein the abnormal object is a human limb capable of being exposed to a disinfection area, and the human limb comprises: a palm, fingers, toes, and a face;
the disinfection method further comprises:
in response to the to-be-disinfected object being not in the effective area of the disinfection lamp, outputting an adjustment prompt information.

25. (canceled)

26. The disinfection method of claim 14, further comprising:
indicating a progress of disinfection.

27. (canceled)

28. A non-transitory storage medium, storing computer-executable instructions thereon, wherein the computer-executable instructions, when executed by a processor, cause the processor to perform operations comprising:
receiving a disinfection instruction; and
controlling, based on the disinfection instruction, a disinfection lamp to output a disinfection light wave of a specific wavelength to an outside to perform disinfection and sterilization, wherein the disinfection lamp is provided on the mobile terminal.
